(19) United States
(12) Patent Application Publication
(10) Pub. No.: US 2012/0034711 A1
(43) Pub. Date: Feb. 9, 2012

(54) Digital immunochromatographic test strip for semi-quantitative detection of aflatoxin B1 and preparation method thereof

(75) Inventors: Peiwu LI, Wuhan (CN); Daohong Zhang, Wuhan (CN); Qi Zhang, Wuhan (CN); Wen Zhang, Wuhan (CN); Xiaoxia Ding, Wuhan (CN); Jun Jiang, Wuhan (CN); Xiaomei Chen, Wuhan (CN)

(73) Assignee: Oil Crops Research Institute, Chinese Academy of Agricultural Science, Wuhan (CN)

(21) Appl. No.: 13/198,260

(22) Filed: Aug. 4, 2011

(30) Foreign Application Priority Data
Aug. 5, 2010 (CN) 201010245094.0

(51) Int. Cl. G01N 33/566; B32B 38/08; G01N 31/22

(52) U.S. Cl. 436/501; 422/424; 156/182

(57) ABSTRACT

The present invention belongs to the field of biological detection. Multi-line immunochromatographic test strip for semi-quantitative detection of aflatoxin B₁ comprises a paperboard, wherein a water-absorbing pad, a detection pad, a gold-labeled pad and a sample pad are adhered sequentially on one surface of the paperboard from top to bottom, wherein each adjacent pads is overlapped and connected, the detection pad uses a nitrocellulose film as a backing pad, the nitrocellulose film is provided with a transverse control line, a test line I, a test line II and a test line III, wherein the control line is coated with a rabbit anti-mouse polyclonal antibody, and the test line I, test line II and test line III are coated with aflatoxin B₁-bovine serum albumin conjugate (AFB₁-BSA), respectively: and the gold-labeled pad is transversely coated with a nanogold-labeled anti-aflatoxin B₁ monoclonal antibody. Said test strip is used for semi-quantitative detection of aflatoxin B₁, and is characterized by quick detection, simple procedure and high sensitivity.

### CROSS-REFERENCE TO RELATED APPLICATIONS

This application claims priority to and the benefit of co-pending Chinese patent application Serial No. 201010245094.0, filed Aug. 5, 2010, which application is incorporated herein by reference in its entirety.

### FIELD OF TECHNOLOGY

The present invention belongs to the field of biological detection, and especially relates to a multi-test-line digital immunochromatographic test strip for semi-quantitative detection of aflatoxin B₁ and preparation method thereof.

### BACKGROUND ART

Aflatoxins are secondary metabolites mainly secreted by Aspergillus flavus and Aspergillus parasiticus, and are a category of natural toxic compounds which can cause serious damages to human and animals. Among the aflatoxins which have been found, aflatoxin B₁ (referred to AFB₁) is the most toxic aflatoxin, and its toxicity, carcinogenicity and frequency of contamination are the most severe among biotoxins.

After contaminating food and animal feed, aflatoxin will directly or indirectly enter the human food chain, and bring risk of health and safety for human, and its danger is proportional to the amount of intake of aflatoxin. Aflatoxin B₁ is found widely in rice, maize, peanut, sesame, soy bean, rapeseed and other agricultural products and other foods such as peanut butter, thus almost all the countries in the world have defined the maximum allowable concentration of aflatoxin B₁ in food and animal feed, and stipulated it as a mandatory standard. Therefore, it is important for ensuring safety of food consumption to improve the detection of aflatoxins, especially aflatoxin B₁, in particular the quick-detection to know and obtain the health information of food and animal feed rapidly.

The conventional aflatoxin B₁ detection techniques in the prior art mainly include thin layer chromatography, precision instruments analysis and immunoassay. The immunoassay method established in recent years overcomes the disadvantages of the first two methods, has advantages such as high specificity, good sensitivity, simplified pre-treatment of sample, low cost, less damage to laboratory personnel and much less environmental pollution, and is suitable for testing in batches on sites. Among the above immunological methods, the immunochromatographic quick-detection technique based on nano-gold is simple, rapid and sensitive, and is suitable for field testing, thus is greatly valuable and has broad prospects for application. However, traditional aflatoxin B₁ immunochromatographic test strip only has one test line, and can only be used for qualitative detection of aflatoxin B₁ in samples, moreover, its sensitivity is low. Therefore, there are still needs to provide a multi-test-line immunochromatographic test strip for aflatoxin B₁ to achieve the semi-quantitative detection of aflatoxin B₁ at high, moderate or low concentration in a sample, and it is of great significance and high value for monitoring aflatoxin B₁ in food and agricultural products.

Usually, a traditional immunochromatographic test strip has only one test line for one analyte and only shows whether a given sample has the analyte at a concentration more or less than the “threshold concentration” represented by the sole test line. While some traditional test strips have multi-test lines, each test line represents a threshold concentration for a different analyte. Here we describe a new immunochromatographic test strip with multi-test lines for the same analyte, and each of the test lines represents a different threshold concentration. Considering its semi-quantitative or quantitative detection feature, the multi-test-line immunochromatographic test strip of the present invention is also sometimes named “digital” immunochromatographic test strip herein.

### SUMMARY OF INVENTION

The technical problem to be solved by the present invention is to provide a multi-test-line digital immunochromatographic test strip for semi-quantitative detection of an analyte, e.g., a biotoxin such as aflatoxin B₁ and preparation methods thereof. The multi-test-line digital immunochromatographic test strip for semi-quantitative detection of aflatoxin is used to semi-quantitatively detect aflatoxin B₁, and has characteristic of quick detection, with a simple procedure and high sensitivity.

As used herein, a multi-test-line digital immunochromatographic test strip is defined as an immunochromatographic test strip with three or more test lines, each of them standing for a specific and different concentration of an analyte (e.g., aflatoxin B₁), and can he used to test at least 4 different concentrations of the analyte on the same strip. Here the term “digital” is used to indicate the ability of the present invention to display the value of the analyte concentration in a relatively narrow range, thereby akin to a reading of an actual value. In various embodiments, the range is set to satisfy how precise the reading needs to be—the narrower the range, the more precise the reading. This quantitative reading is made possible because there are three or more test lines on the immunochromatographic test strip disclosed herein and each of them represents a different threshold concentration, which means a user can deduce a concentration figure on such a digital immunochromatographic test strip.

To solve said technical problems encountered in the prior art, the present invention provides embodiments as following:

Multi-test-line digital immunochromatographic test strip for semi-quantitative detection of an analyte, e.g., aflatoxin B₁ (see FIG. 1), comprising a backing (e.g., paperboard), wherein a water-absorbing pad, a detection pad, a marker-labeled pad and a sample pad are adhered or attached sequentially on one side of said backing from top to bottom, wherein each pad is in direct contact with its neighboring pad or pads. In one embodiment, the pads overlap with each other and are connected.

The detection pad may use a nitrocellulose film as a hacking pad, and the nitrocellulose film is provided with a transverse control line, and a plurality of test lines (e.g., a test line I, a test line II and a test line III) from top to bottom. In one feature, the test line I, test line II and test line III are each coated with aflatoxin B₁-bovine serum albumin (AFB₁-BSA) conjugate. In one embodiment, the control line and the test lines traverse the detection pad in a direction perpendicular to a longitudinal axis of the test strip. The control line provides positive control for the test strip and can be viewed as a quality control line.

The marker-labeled pad is coated with a labeled primary antibody against the analyte, e.g., it can be transversely coated through spraying with a nanogold-labeled anti-aflatoxin B₁ monoclonal antibody. In that case, the marker-labeled pad can be called a gold-labeled pad. The control line in the detection pad is coated with a secondary antibody against the primary antibody provided in the marker-labeled pad. In an embodiment where the primary antibody used in the marker-labeled pad is derived from mouse, the control line in the detection pad may be coated with a rabbit anti-mouse polyclonal antibody.

According to the embodiment mentioned above, the water-absorbing pad has the length of 16˜18 mm and the width of 2˜4 mm: the detection pad has the length of 25˜30 mm and the width of 2˜4 mm; the gold-labeled pad has the length of 6˜9 mm and the width of 2˜4 mm; the sample pad has the length of 12˜18 mm and the width of 2˜4 mm, and overlapped parts of each adjacent pad have the length of 1˜3 mm.

According to the embodiment mentioned above, the water-absorbing pad is made of a water-absorbing paper. The sample pad is where a test sample is loaded.

According to the embodiment mentioned above, the distances between the test line I, test line II and test line III and the upper border of the nitrocellulose film on the detection pad are 11˜17 mm, 13˜19 mm and 15˜21 mm, respectively, and the distances between each two adjacent test lines are at least 2 mm; and the distance between the control line and the test line I is 5˜11 mm.

According to the embodiment mentioned above, the coating amount of aflatoxin B₁-bovine serum albumin (AFB₁-BSA) conjugate required on per cm of the test line I, test line II and test line III on the detection pad are 1201˜600 ng, 40˜200 ng and 20˜100 ng, respectively, and the coating amount of rabbit anti-mouse polyclonal antibody required on per cm of the control line is 200˜500 ng.

According to the embodiment mentioned above, the particle diameter of the nanogold used in said gold-labeled pad is 15˜20 nm.

According to the embodiment mentioned above, the amount of nanogold-labeled anti-aflatoxin B1 monoclonal antibody required on per cm of spraying length on the gold-labeled pad is 60˜216 ng.

The present invention provides a method for preparation of the high sensitive digital immunochromatographic test strip for semi-quantitative detection of aflatoxin B₁, which comprises:

(1) preparation of the water-absorbing pad,

wherein the water-absorbing pad is obtained by cutting a water-absorbing paper;

(2) preparation of the detection pad, including the following steps:

Coating of the test lines:

wherein commercially available aflatoxin B₁-bovine serum albumin conjugate (AFB₁-BSA) is used to prepare 0.1˜0.5 mg mL⁻¹ of coating solution A, and the nitrocellulose film is coated transversely with the coating solution A along the positions of 11˜17 mm, 13˜19 mm and 15˜21 mm from the upper border of said film by spot-spraying, resulting in the test line I, test line II and test line III, the distances between each test line are at least 2 mm, and the coating amount of aflatoxin B₁-bovine serum albumin conjugate (AFB₁-BSA) required on per cm of the test line I, test line II and test line III is 120˜600 ng, 40˜200 ng, and 20˜100 ng, respectively, then said film is dried for 8˜20 minutes at 37˜40° C.;

Coating of the control line:

wherein the rabbit anti-mouse polyclonal antibody is used to prepare 0.4˜0.6 mg mL-1 of coating solution B; and the nitrocellulose film is coated transversely with coating solution B at the position of 5˜11 mm from the test line I on the nitrocellulose film by spot-spraying to obtain the control line, and the coating amount of rabbit anti-mouse polyclonal antibody required on per cm of the control line is 200˜500 ng, then said film is dried for 8˜20 minutes at 37˜40° C.;

(3) preparation of the sample pad

wherein the fiberglass film is put into blocking solution A to be soaked, then taken out of the solution and dried for 10˜16 h at 37˜40° C. to obtain the sample pad, and placed in a desiccator/drier to store at room temperature;

(4) preparation of the gold-labeled pad

wherein the nanogold-labeled anti-aflatoxin B₁ monoclonal antibody solution is transversely sprayed onto the sample pad by spot-spraying, the amount of nanogold-labeled anti-aflatoxin B₁ monoclonal antibody required on per cm of spraying length is 60˜216 ng, and after lyophilization under vacuum for 2˜6 h, the gold-labeled pad is prepared and placed in a desiccator to store at room temperature;

(5) assembly of the multi-test-line digital immunochromatographic test strip for semi-quantitative detection of aflatoxin B1

wherein a water-absorbing pad, a detection pad, a gold-labeled pad and a sample pad are adhered sequentially on one surface of said paperboard from top to bottom, wherein each adjacent pads are overlapped and connected, and the length of each overlapped part is 1˜3 mm, thus the multi-test-line digital immunochromatographic test strip for semi-quantitative detection of aflatoxin B₁ is prepared (see FIG. 1 and FIG. 2).

According to the embodiment mentioned above, the coating solution A comprises 10˜50 mg of commercially available aflatoxin B₁-bovine serum albumin conjugate (AFB₁-BSA), 1˜2 g bovine serum albumin, 1˜2 g sucrose, 0.02˜0.05 g sodium azide, 0.8 g sodium chloride, 0.29 g disodium hydrogen phosphate dodecahydrate, 0.02 g potassium chloride, 0.02 g potassium dihydrogen phosphate, to which water is added to reach 100 mL of final volume;

The coating solution B comprises 50 mg rabbit anti-mouse polyclonal antibody, 0.02˜0.05 g sodium azide, 0.8 g sodium chloride, 0.29 g disodium hydrogen phosphate dodecahydrate, 0.02 g potassium chloride, 0.02 g potassium dihydrogen phosphate, to which water is added to reach 100 mL of final volume.

According to the embodiment mentioned above, the blocking solution A comprises 1˜2 g bovine serum albumin, 0.1˜0.2 mL Triton X-100, 0.3 g polyvinylpyrrolidone, 2˜5 g sucrose, 0.02˜0.05 g sodium azide, 0.8 g sodium chloride, 0.29 g disodium hydrogen phosphate dodecahydrate, 0.02 g potassium chloride, 0.02 g potassium dihydrogen phosphate, to which water is added to reach 100 mL of final volume.

According to the embodiment mentioned above, the method for preparation of nanogold-labeled anti-aflatoxin B₁ monoclonal antibody solution is described as follows: 50.0 mL of commercially available nanogold solution is taken with the mass concentration of 0.01%, and the pH of the solution is adjusted to 5.5; 2 mL of 0.1 mg mL⁻¹ anti-aflatoxin B₁ monoclonal antibody aqueous solution is added slowly with stirring and the solution is further stirred for 30 min; 10% (w/w) bovine serum albumin aqueous solution is added until the final concentration of bovine serum albumin (BSA) is 1% (w/w), and the solution is further stirred for 30 min; after standing at 4° C. for 2 h, the solution is centrifuged at 1500 rpm for 15 min. The supernatant is removed and the pellet is discarded: the supernatant obtained is centrifuged at 12000 rpm for 30 min, and the supernatant is discarded, then 50 mL label-washing preservation solution is added and the resulting solution is centrifuged at 12000 rpm for 30 min again. The supernatant is discarded, and the precipitate obtained is resuspended with label-washing preservation solution to obtain the concentrated solution with the volume of 5 mL, which is stored at 4° C. wherein the mass concentration of the nanogold-labeled anti-aflatoxin B₁ monoclonal antibody solution is 0.04 mg mL⁻¹.

Said label-washing preservation solution is prepared by mixing 2.0 g polyethylene glycol-20000 (PEG-20000), 0.2 g sodium azide and 0.1235 g boric acid, to which water is added to 1000 mL, followed by filtration through 0.22 μm filter membrane.

The use of said multi-test-line digital immunochromatographic test strip for semi-quantitative detection of aflatoxin B₁: the test sample grinded finely is weighed, to which methanol aqueous solution with the concentration of 60˜80% (v/v) is added, and the m/v ratio of the test sample and methanol solution is 2 g mL⁻¹. The solution is well-mixed and extracted with sonication in 50˜60° C. water-bath for 5˜10 min, then left for 5˜10 min. The supernatant (i.e. the extract) is diluted to 1:2.5 with water, resulting in that the final concentration of methanol in the dilution is 24˜32%. 100 μL diluted sample solution used as the test solution is added dropwise to the sample pad of the multi-test-line digital immunochromatographic test strip for semi-quantitative detection of aflatoxin B₁. Said test strip is used as the test strip for detection. In the meantime, 100 μL of water is used as the negative control solution and added dropwise to the sample pad of another multi-test-line digital immunochromatographic test strip for semi-quantitative detection of aflatoxin B₁, and said test strip is used as the control test strip. The results are read out after 15 min.

Result evaluation: (1) positive: the control line of the test strip for detection of test sample shows a red line, if the color of test line I among the three test lines is slightly lighter than that of the control test strip, and those of test line II and test line III are substantially the same as the control test strip, the content of aflatoxin B₁ in the sample is between 0.625 and 1.25 ng g⁻¹; if the test line I among the three test lines does not show red, and the colors of test line II and test line III are substantially the same as the control test strip, the content of aflatoxin B₁ in the sample is 1.25 ng g⁻¹: if the test line I among the three test lines does not show red, the color of test line II is lighter than that of the control test strip, and the color of test line III is substantially the same as the control test strip, the content of aflatoxin B₁ in the sample is between 1.25 and 2.5 ng g⁻¹: if the test line I and II of the three test lines do not show red, and the color of test line III is substantially the same as the control test strip, the content of aflatoxin B, in the sample is 2.5 ng g⁻¹: if the test line I and II of the three test lines do not show red, and the color of test line III is lighter than that of the control test strip, the content of aflatoxin B₁ in the sample is between 2.5 and 10 ng g⁻¹; if all the three test lines do not show red, the content of aflatoxin B₁ in the sample is not less than 10 ng g⁻¹. (2) negative: the control line of the test strip for detection of test sample shows a red line, and the colors of the three test lines are close to those of the control test strip, then it is a negative result, which indicates the content of aflatoxin B₁ in the sample is less than 0.625 ng g^{31 1}. (3) Null: no matter the test lines of the test strip for detection of test sample show red lines or not, the test strip is considered as null as long as the control line does not exhibit a red line.

The working principle of the test strip is as following: when the sample solution is added to the sample pad in the bottom of the test strip, it moves toward the water-absorbing pad due to capillarity, and when it moves to the gold-labeled pad, the nanogold-labeled anti-aflatoxin B₁ monoclonal antibody is dissolved. If the sample contains aflatoxin B₁, aflatoxin B₁ will bind to the nanogold-labeled anti-aflatoxin B₁ monoclonal antibody on the gold-labeled pad and move upward together, when they reach the three test lines fixed with antigen, the antigen and aflatoxin B₁ will competitively bind to the finite antigen binding sites on the nanogold-labeled anti-aflatoxin B₁ monoclonal antibody. The more aflatoxin B₁ contained in the sample, the less nanogold-labeled anti-aflatoxin B₁ monoclonal antibody the antigen on the test lines can bind, the less colored line generates, and the lighter the color is. If the amount of the nanogold-labeled anti-aflatoxin B₁ monoclonal antibody bound by the antigen is less than a certain value, no red line can be exhibited by the test lines. No matter the sample contains aflatoxin B₁ or not, nanogold-labeled anti-aflatoxin B₁ monoclonal antibody which is not captured by the test lines or the complex of the nanogold-labeled anti-aflatoxin B₁ monoclonal antibody and aflatoxin B₁ will move on to the control line, and bind to the second antibody thereon, thus being enriched and showing its color. Therefore, no matter the sample contains aflatoxin B₁ or not, the control line will be colored. When the sample does not contain aflatoxin B₁ (i.e. negative), the strip appears four red lines, i.e. the control line and three test lines; when the sample contains certain amount of aflatoxin B₁ (i.e. positive), six possible results may be observed from the test strip after completion of detection: (1) the test line I is pale red, both test line II and test line III are red, and the control line is red; (2) the test line I does not show red, both test line II and test line III are red, and the control line is red; (3) the test line I does not show red, the test line II is pale red, the test line III is red, and the control line is red; (4) both the test line I and the test line II do not show red, the test line III is red, and the control line is red; (5) both the test line I and the test line II do not show red, the test line III is pale red, and the control line is red; (6) none of the test line I, test line II and test line III is colored, and the control line is red; if the control line does not show red, the test strip is null.

By application of the strip, four or more than four concentrations of aflatoxin B₁ can be detected on the same strip.

The beneficial effects brought by the present invention include:

(1) semi-quantitative detection of aflatoxin B₁. The multi-test-line digital immunochromatographic test strip for semi-quantitative detection of aflatoxin B₁ according to the present invention comprises three test lines, and can be used for semi-quantitative detection of aflatoxin B₁ at high, moderate or low concentration, which is greatly valuable for practical application.

(2) Simplified pre-treatment of sample. To pre-treat samples, one only needs to add methanol to the sample that has bee grinded finely, and to extract with ultrasound technology. After letting the extract stand to settle, the supernatant removed and diluted for detection. Thus, the entire process of sample pre-treatment is simple and fast.

(3) Simple operation procedure. Using the multi-test-line digital immunochromatographic test strip for semi-quantitative detection of aflatoxin B₁, one only needs to add, drop by drop, the extract solution from the sample to the sample pad of test strip, which is a one-step operation and do not need any professionals, thus the procedure is simple and convenient.

(4) Being friendly to the environment. The detection process does not require the standard solution of aflatoxin B₁ as a positive control. The present invention provides a test strip which does not need to add the standard solution of aflatoxin B₁ as a positive control and only needs water as a negative control when detecting samples, thus avoiding any second-time pollution by aflatoxin B₁.

(5) High sensitivity. The lowest detectable limit of the aflatoxin B₁ in a sample using the semi-quantitative multi-test-line digital immunochromatographic test strip of the present invention is 0.625 ng g⁻¹, which is lower than the lowest detection limit of aflatoxin B₁ in food regulated by the EU.

### BRIEF DESCRIPTION OF DRAWINGS

FIG. 1 is a diagram of the multi-test-line digital immunochromatographic test strip for semi-quantitative detection of aflatoxin B₁ according to the present invention, wherein 1: paperboard; 2: water-absorbing pad; 3: detection pad; 4: gold-labeled pad; 5: sample pad; 6: control line; 7: test line I; 8: test line II; 9: test line III.

FIG. 2 is a side view of multi-test-line digital immunochromatographic test strip for semi-quantitative detection of aflatoxin B₁ according to the present invention in side view, wherein 1: paperboard; 2: water-absorbing pad; 3: detection pad; 4: gold-labeled pad; 5: sample pad.

FIG. 3 is a representative diagram of the result of example 1, wherein 1: control test strip; 2: test strip for detection; 3: control line; 4: test line I; 5: test line II; 6: test line III.

FIG. 4 is a representative diagram of the result of example 2, wherein 1: control test strip; 2: test strip for detection; 3: control line: 4: test line I; 5: test line II; 6: test line III.

FIG. 5 is a representative diagram of the result of example 3, wherein 1: control test strip: 2: test strip for detection; 3: control line; 4: test line I; 5: test line II; 6: test line III.

### DETAILED DESCRIPTION OF THE INVENTION

### EXAMPLES 1-3

### Preparation of High Sensitive Digital Immunochromatographic Test Strip for Semi-Quantitative Detection of Aflatoxin and Use Thereof

Commercially available anti-aflatoxin B₁ monoclonal antibody A9555 was used in the following examples 1-3. However, the antibodies which can be used in the present invention are not limited to the specific antibody used in the following examples, other aflatoxin B₁ antibodies are also applicable, and the difference between them only exists in detection sensitivity.

### Example 1

The method for preparation of high sensitive digital immunochromatographic test strip for semi-quantitative detection of aflatoxin B₁ comprised the following steps:

(1) preparation of the water-absorbing pad

The water-absorbing pad was obtained by cutting a water-absorbing paper into the size of 16 mm×3 mm;

(2) preparation of the detection pad

Coating of the test lines:

Commercially available aflatoxin B₁-bovine serum albumin conjugate (AFB₁-BSA) was used to prepare 0.1 mg mL⁻¹ of coating solution A, and the nitrocellulose film was coated transversely with coating solution A along the positions of 13 mm, 15 mm and 17 mm from the upper border of said film by spot-spraying, resulting in the test line I, test line II and test line III, and the coating amount of aflatoxin B₁-bovine serum albumin conjugate (AFB₁-BSA) required on per cm of the test line I, test line II and test line III was 120 ng, 40 ng and 20 ng, respectively, then said film was dried for 8 minutes at 37° C.;

The coating solution A included 10 mg of commercially available aflatoxin B₁-bovine serum albumin conjugate (AFB₁-BSA), 2 g bovine serum albumin, 2 g sucrose, 0.02 g sodium azide, 0.8 g sodium chloride, 0.29 g disodium hydrogen phosphate dodecahydrate, 0.02 g potassium chloride, 0.02 g potassium dihydrogen phosphate, to which water was added to reach 100 mL of final volume.

Coating of the control line:

The rabbit anti-mouse polyclonal antibody was used to prepare 0.4 mg mL⁻¹ of coating solution B; and the nitrocellulose film was coated transversely with coating solution B at the position of 5 mm from the test line I on the nitrocellulose film by spot-spraying to obtain the control line, and the coating amount of rabbit anti-mouse polyclonal antibody required on per cm of the control line was 200 ng, then said film was dried for 8 minutes at 37° C.;

The coating solution B included 50 mg rabbit anti-mouse polyclonal antibody, 0.02 g sodium azide, 0.8 g sodium chloride, 0.29 g disodium hydrogen phosphate dodecahydrate, 0.02 g potassium chloride, 0.02 g potassium dihydrogen phosphate, to which water was added to reach 100 mL of final volume.

The nitrocellulose film had a length of 25 mm and a width of 3 mm.

(3) preparation of the sample pad

The fiberglass film was cut into the size of 15 mm×3 mm and put into blocking solution A to be soaked, then taken out of the solution and dried for 10 h at 37° C. to obtain the sample pad, and placed in a desiccator to store at room temperature;

The blocking solution A included 2 g bovine serum albumin; 0.1 mL Triton X-100, 0.3 g polyvinylpyrrolidone, 2.5 g sucrose, 0.02 g sodium azide, 0.8 g sodium chloride, 0.29 g disodium hydrogen phosphate dodecahydrate, 0.02 g potassium chloride, 0.02 g potassium dihydrogen phosphate, to which water was added to reach 100 mL of final volume.

(4) preparation of the gold-labeled pad

The sample pad was cut into the size of 8 mm×3 mm, and the nanogold-labeled anti-aflatoxin B₁ monoclonal antibody solution was transversely sprayed onto the sample pad by spot-spraying, the amount of nanogold-labeled anti-aflatoxin B₁ monoclonal antibody required on per cm of spraying length was 192 ng. After lyophilization under vacuum for 6 h, the gold-labeled pad was prepared and placed in a desiccator to store at room temperature:

The method for preparation of 0.04 mg mL⁻¹ nanogold-labeled anti-aflatoxin B₁ monoclonal antibody solution is described as follows: 50.0 mL of commercially available nanogold solution was taken with the mass concentration of 0.01%, and the pH of the solution was adjusted to 5.5 with 0.1 mol L⁻¹ potassium carbonate aqueous solution; 2 mL of 0.1 mg mL⁻¹ anti-aflatoxin B₁ monoclonal antibody aqueous solution was added slowly with stirring and the solution was further stirred for 30 min; 10% (w/w) bovine serum albumin aqueous solution was added until the final concentration of bovine serum albumin is 1% (w/w), and the solution was further stirred for 30 min; after standing at 4° C. for 2 h, it was centrifuged at 1500 rpm for 15 min. The supernatant was removed and the pellet is discarded; the supematant obtained was centrifuged at 12000 rpm for 30 min, and the supernatant was discarded, then 50 mL label-washing preservation solution was added and the resulting solution was centrifuged at 12000 rpm for 30 min again. The supernatant was discarded, and the precipitate obtained was resuspended with label-washing preservation solution to obtain the concentrated solution with the volume of 5.0 mL, which was stored at 4° C. for use, wherein the mass concentration of the nanogold-labeled anti-aflatoxin B₁ monoclonal antibody solution was 0.04 mg mL⁻¹;

The particle diameter of the nanogold in said nanogold solution was 15 nm;

Said 0.1 mol L⁻¹ potassium carbonate solution was obtained as follows: 13.8 g potassium carbonate was dissolved in purified water and diluted to 1000 mL of final volume, then filtered by 0.22 μm filter membrane; said 0.1 mg mL⁻¹ anti-aflatoxin B₁ monoclonal antibody solution was obtained as follows: 1 mg of commercially available anti-aflatoxin B₁ monoclonal antibody was diluted with water to 10 mL of final volume; said 10% bovine serum albumin aqueous solution was obtained as follows: 10 g bovine serum albumin was diluted with water to 100 mL, then filtered by 0.22 μm filter membrane: said label-washing preservation solution was obtained by mixing 2.0 g PEG-20000, 0.2 g sodium azide and 0.1235 g boric acid, to which water was added to a final volume of 1000 mL, followed by filtration through 0.22 μm filter membrane.

(5) assembly of the multi-test-line digital immunochromatographic test strip for semi-quantitative detection of aflatoxin B₁

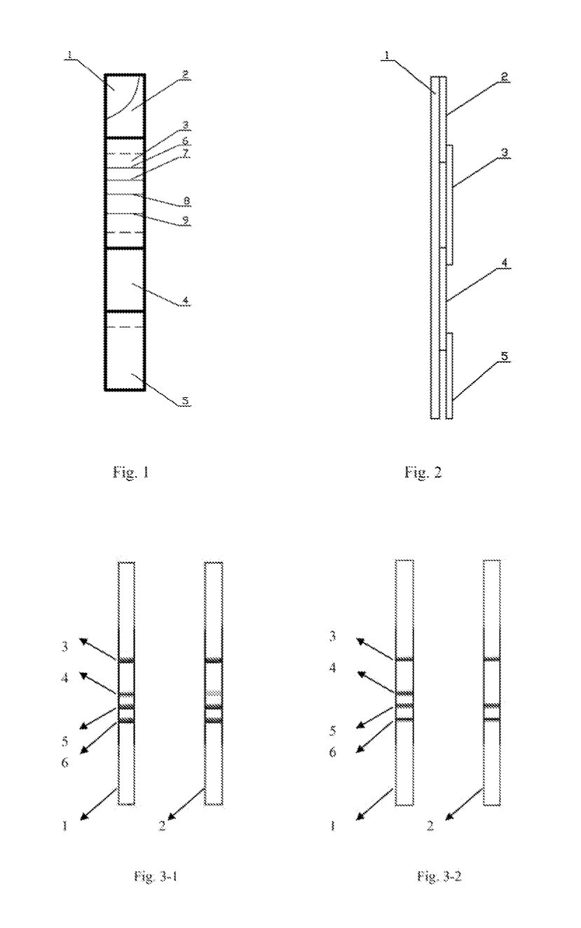

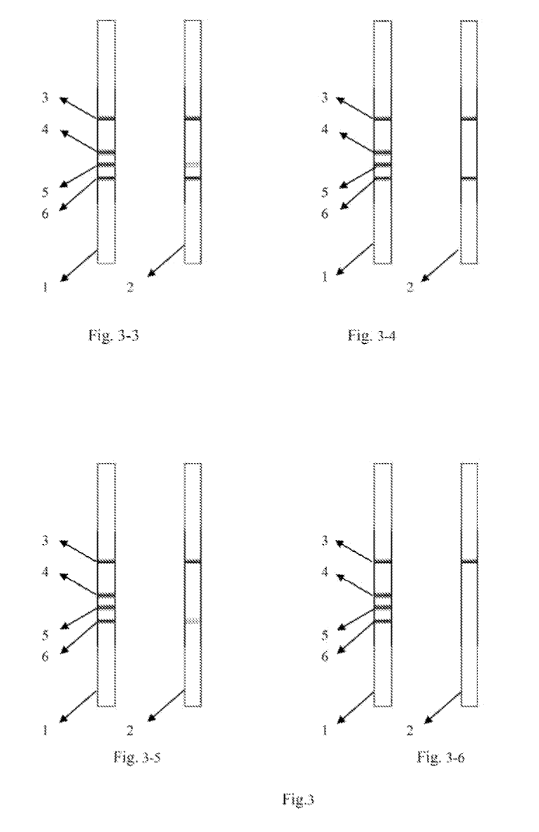

A water-absorbing pad, a detection pad, a gold-labeled pad and a sample pad were adhered sequentially on one surface of said paperboard from top to bottom, wherein each adjacent pads were overlapped and connected, and the length of each overlapped part was 1 mm, thus the multi-test-line digital immunochromatographic test strip for detection of aflatoxin B₁ was prepared (see FIG. 1 and FIG. 2).

The method for use of the multi-test-line digital immunochromatographic test strip for semi-quantitative detection of aflatoxin B₁ prepared above is described as follows: the test samples of 1-6 which had been grinded finely were weighed, to which methanol aqueous solution with the concentration of 60˜80% (v/v) was added, and the m/v ratio of the test sample and methanol solution was 2 g mL⁻¹. The solution was well-mixed and extracted with ultrasound at 50° C. water bath for 8 min, then left for 10 min. The supernatant (i.e. the extract) was diluted to 1:2.5 with water, resulting in a final concentration of methanol in the dilution of 32%. 100 μL diluted sample solution used as the test solution was added dropwise to the sample pad of the multi-test-line digital immunochromatographic test strip for semi-quantitative detection of aflatoxin B₁. Said test strip was used as the test strip for detection. In the meantime, 100 μL of water was used as the negative control solution and added dropwise to the sample pad of another multitest-line digital immunochromatographic test strip for semi-quantitative detection of aflatoxin B₁, which was used as the control test strip. The results were read out after 15 min.

Result evaluation: the control line of the test strip for detection of the 1st test sample showed a red line, while the color of test line I among the three test lines was slightly lighter than that of the control test strip, and those of test line II and test line III were substantially the same as the control test strip, thus it was a positive result, which indicates that the content of aflatoxin B₁ in the sample is between 0.625 and 1.25 ng g⁻¹, see FIG. 3-1;

The control line of the test strip for detection of the 2nd test sample showed a red line, while the test line I among the three test lines did not show red, and the colors of test line II and test line III were substantially the same as the control test strip, thus it was a positive result, which indicated that the content of aflatoxin B₁ in the sample was 1.25 ng g⁻¹, see FIG. 3-2:

The control line of the test strip for detection of the 3rd test sample showed a red line, while the test line I among the three test lines did not show red, the color of test line II was lighter than that of the control test strip, and the color of test line III was substantially the same as the control test strip, thus it was a positive result, which indicated that the content of aflatoxin B₁ in the sample was between 1.25 and 2.5 ng g⁻¹: see FIG. 3-3;

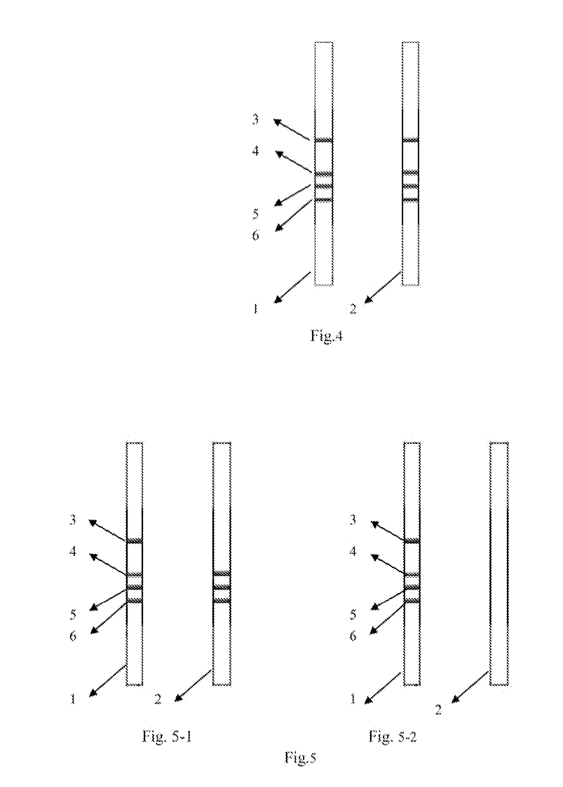

The control line of the test strip for detection of the 4th test sample showed a red line, while the test line I and II of the three test lines did not show red, and the color of test line III was substantially the same as the control test strip, thus it was a positive result, which indicated that the content of aflatoxin B₁ in the sample was 2.5 ng g⁻¹, see FIG. 3-4:

The control line of the test strip for detection of the 5th test sample showed a red line, while the test line I and II of the three test lines did not show red, and the color of test line III was lighter than that of the control test strip, thus it was a positive result, which indicated that the content of aflatoxin B₁ in the sample was between 2.5 and 10 ng g⁻¹, see FIG. 3-5;

The control line of the test strip for detection of the 6th test sample showed a red line, while all the three test lines did not show red, thus it was a positive result, which indicated that the content of aflatoxin B₁ in the sample was over 10 ng g⁻¹, see FIG. 3-6.

### Example 2

The method for preparation of the high sensitive digital immunochromatographic test strip for semi-quantitative detection of aflatoxin B₁ included the following steps:

(1) preparation of the water-absorbing pad

The water-absorbing pad was obtained by cutting a water-absorbing paper into the size of 17 mm×2 mm:

(2) preparation of the detection pad

Coating of the test lines:

Commercially available aflatoxin B1-bovine serum albumin conjugate (AFB₁-BSA) was used to prepare 0.25 mg mL⁻¹ of coating solution A, and the nitrocellulose film was coated transversely with coating solution A along the positions of 15 mm, 17 mm, 19 mm from the upper border of said film by spot-spraying, resulting in the test line I, test line II and test line III, and the coating amount of aflatoxin B₁-bovine serum albumin conjugate (AFB₁-BSA) required on per cm of the test line I, test line II and test line III was 300 ng, 100 ng and 50 ng, respectively, then said film is dried for 10 min at 38° C.;

The coating solution A comprises 25 mg of commercially available aflatoxin B₁-bovine serum albumin conjugate (AFB₁-BSA), 1 g bovine serum albumin, 2 g sucrose, 0.03 g sodium azide, 0.8 g sodium chloride, 0.29 g disodium hydrogen phosphate dodecahydrate, 0.02 g potassium chloride, 0.02 g potassium dihydrogen phosphate, to which water was added to reach 100 mL of final volume.

Coating of the control line:

The rabbit anti-mouse polyclonal antibody was used to prepare 0.5 mg mL⁻¹ of coating solution B; and the nitrocellulose film was coated transversely with coating solution B at the position of 7 mm from the test line I on the nitrocellulose film by spot-spraying to obtain the control line, and the coating amount of rabbit anti-mouse polyclonal antibody required on per cm of the control line was 300 ng, then said film was dried for 15 min at 38° C.;

The coating solution B included 50 mg rabbit anti-mouse polyclonal antibody, 0.03 g sodium azide, 0.8 g sodium chloride, 0.29 g disodium hydrogen phosphate dodecahydrate, 0.02 g potassium chloride, 0.02 g potassium dihydrogen phosphate, to which water was added to reach 100 mL for the final volume.

The nitrocellulose film had a length of 28 mm and a width of 2 mm.

(3) preparation of the sample pad

The fiberglass film was cut into the size of 16 mm×2 mm and put into blocking solution A to be soaked, then taken out of the solution and dried for 12 h at 38° C. to obtain the sample pad, and placed in a desiccator to store at room temperature;

The blocking solution A comprises 1˜2 g bovine serum albumin, 0.1˜0.2 mL Triton X-100, 0.3 g polyvinylpyrrolidone, 2˜5 g sucrose, 0.02˜0.05 g sodium azide, 0.8 g sodium chloride, 0.29 g disodium hydrogen phosphate dodecahydrate, 0.02 g potassium chloride, 0.02 g potassium dihydrogen phosphate, to which water was added to reach 100 mL of final volume.

(4) preparation of the gold-labeled pad

The sample pad was cut into the size of 6 mm×2 mm, and the nanogold-labeled anti-aflatoxin B₁ monoclonal antibody solution was transversely sprayed onto the sample pad by spot-spraying, the amount of nanogold-labeled anti-aflatoxin B₁ monoclonal antibody required on per cm of spraying length was 120 ng. After lyophilization under vacuum for 5 h, the gold-labeled pad was prepared and placed in a desiccator to store at room temperature:

The method for preparation of nanogold-labeled anti-aflatoxin B₁ monoclonal antibody solution is described as follows: 50.0 mL of commercially available nanogold solution was taken with the mass concentration of 0.01%, and the pH of the solution was adjusted to 5.5 with 0.1 mol L⁻¹ potassium carbonate aqueous solution; 2 mL of 0.1 mg mL⁻¹ anti-aflatoxin B₁ monoclonal antibody aqueous solution was added slowly with stirring and the solution was further stirred for 30 min: 10% (w/w) bovine serum albumin aqueous solution was added until the final concentration of bovine serum albumin is 1% (w/w), and the solution was further stirred for 30 min; after standing at 4° C. for 2 h, it was centrifuged at 1500 rpm for 15 min. The supernatant was removed and the pellet was discarded; the supernatant obtained was centrifuged at 12000 rpm for 30 min, and the supernatant was discarded, then 50 mL label-washing preservation solution was added and the resulting solution was centrifuged at 12000 rpm for 30 min again. The supernatant was discarded, and the precipitate obtained was resuspended with label-washing preservation solution to obtain the concentrated solution with the volume of 5.0 mL, which was stored at 4° C. for later use, wherein the mass concentration of the nanogold-labeled anti-aflatoxin B₁ monoclonal antibody solution was 0.04 mg mL⁻¹;

The particle diameter of the nanogold in said nanogold solution was 18 nm;

Said 0.1 mol L-1 potassium carbonate solution was obtained as follows: 13.8 g potassium carbonate was dissolved in purified water and diluted to 1000 mL of filial volume, then filtered by 0.22 μm filter membrane; said 0.1 mg mL⁻¹ anti-aflatoxin B₁ monoclonal antibody solution was obtained as follows: 1 mg of commercially available anti-aflatoxin B₁ monoclonal antibody was diluted with water to 10 mL of final volume; said 10% bovine serum albumin aqueous solution was obtained as follows: 10 g bovine serum albumin was diluted with water to 100 mL, then filtered by 0.22 μm filter membrane; said label-washing preservation solution was obtained by mixing 2.0 g PEG-20000, 0.2 g sodium azide and 0.1235 g boric acid, to which water is added to 1000 mL, followed by filtration through 0.22 μm filter membrane.

(5) assembly of the multi-test-line digital immunochromatographic test strip for semi-quantitative detection of aflatoxin B₁

A water-absorbing pad, a detection pad, a gold-labeled pad and a sample pad were adhered sequentially on one surface of said paperboard from top to bottom, wherein each adjacent pads were overlapped and connected, and the length of each overlapped part was 2 mm, thus the multi-test-line digital immunochromatographic test strip for semi-quantitative detection of aflatoxin B₁ was prepared (see FIG. 1 and FIG. 2).

The method for use of the multi-test-line digital immunochromatographic test strip for semi-quantitative detection of aflatoxin B₁ is described as follows: the test samples which had been grinded finely were weighed, to which methanol aqueous solution with the concentration of 60˜80% (v/v) was added, and the m/v ratio of the test sample and methanol solution was 2 g mL⁻¹. The solution was well-mixed and extracted with sonication at 50° C. water bath for 5 min, then left for 5 min. The supernatant (i.e. the extract) was diluted to 1:2.5 with water, resulting in that the final concentration of methanol in the dilution was 28%. 100 μL diluted sample solution used as the test solution was added dropwise to the sample pad of the multi-test-line digital immunochromatographic test strip for semi-quantitative detection of aflatoxin B₁. Said test strip was used as the test strip for detection. In the meantime, 100 μL of water was used as the negative control solution and added dropwise to the sample pad of another multi-test-line digital immunochromatographic test strip for semi-quantitative detection of aflatoxin B₁, which was used as the control test strip. The results were read out after 15 min.

Result evaluation: The control line of the test strip for detection of the test sample showed a red line, while the colors of the three test lines were close to those of the control test strip, thus it was judged as a negative result, see FIG. 4, which indicated the content of aflatoxin B₁ in the sample was less than 0.625 ng⁻.

### Example 3

The method for preparation of high sensitive digital immunochromatographic test strip for semi-quantitative detection of aflatoxin B₁ comprised the following steps:

(1) preparation of the water-absorbing pad

The water-absorbing pad was obtained by cutting a water-absorbing paper into the size of 18 mm×4 mm;

(2) preparation of the detection pad

Coating of the test lines:

Commercially available aflatoxin B₁-bovine serum albumin conjugate (AFB₁-BSA) was used to prepare 0.5 mg mL⁻¹ of coating solution A, and the nitrocellulose film was coated transversely with coating solution A alone the positions of 17 mm, 19 mm, 21 mm from the upper border of said film by spot-spraying, resulting in the test line I, test line II and test line III, and the coating amount of aflatoxin B₁-bovine serum albumin conjugate (AFB₁-BSA) required on per cm of the test line I, test line II and test line III is 600 ng, 200 ng and 100 ng, respectively, then said film was dried for 10 minutes at 39° C.;

The coating solution A included 50 mg of commercially available aflatoxin B₁-bovine serum albumin conjugate (AFB₁-BSA), 1.5 g bovine serum albumin, 1.5 g sucrose, 0.02 g sodium azide, 0.8 g sodium chloride, 0.29 g disodium hydrogen phosphate dodecahydrate, 0.02 g potassium chloride, 0.02 g potassium dihydrogen phosphate, to which water was added to reach 100 mL of final volume.

Coating of the control line:

Rabbit anti-mouse polyclonal antibody was used to prepare 0.6 mg mL⁻¹ of coating solution B: and the nitrocellulose film was coated transversely with coating solution B at the position of 9 mm from the test line I on the nitrocellulose film by spot-spraying to obtain the control line, and the coating amount of rabbit anti-mouse polyclonal antibody required on per cm of the control line was 500 ng, then said film was dried for 10 minutes at 39° C.;

The coating solution B comprises 50 mg rabbit anti-mouse polyclonal antibody, 0.02 g sodium azide, 0.8 g sodium chloride, 0.29 g disodium hydrogen phosphate dodecahydrate, 0.02 g potassium chloride, 0.02 g potassium dihydrogen phosphate, to which water was added to reach 100 mL of final volume.

The nitrocellulose film had a length of 30 mm and a width of 4 mm.

(3) preparation of the sample pad

The fiberglass film was cut into the size of 17 mm×4 mm and put into blocking solution A to be soaked, then taken out of the solution and dried for 10 h at 39° C. to obtain the sample pad, and placed in a desiccator to store at room temperature;

The blocking solution A included 1.5 g bovine serum albumin, 0.15 mL Triton X-100, 0.3 g polyvinylpyrrolidone, 4 g sucrose, 0.02 g sodium azide, 0.8 g sodium chloride, 0.29 g disodium hydrogen phosphate dodecahydrate, 0.02 g potassium chloride, 0.02 g potassium dihydrogen phosphate, to which water was added to reach 100 mL of final volume.

(4) preparation of the gold-labeled pad

The sample pad is cut into the size of 6 mm×4 mm, and the nanogold-labeled anti-aflatoxin B₁ monoclonal antibody solution was transversely sprayed onto the sample pad by spot-spraying, the amount of nanogold-labeled anti-aflatoxin B₁ monoclonal antibody required on per cm of spraying length was 72 ng. After lyophilization under vacuum for 2 h, the gold-labeled pad was prepared and placed in a desiccator to store at room temperature;

The particle diameter of the nanogold in said nanogold solution was 20 nm;

The method for preparation of nanogold-labeled anti-aflatoxin B₁ monoclonal antibody solution is described as follows: 50.0 mL of commercially available nanogold solution was taken with the concentration of 0.01%, and the pH of the solution was adjusted to 5.5 with 0.1 mol L⁻¹ potassium carbonate aqueous solution; 2 mL of 0.1 mg mL⁻¹ anti-aflatoxin B₁ monoclonal antibody aqueous solution was added slowly with stirring and the solution was further stirred for 30 min; 10% bovine serum albumin aqueous solution was added until the final concentration of bovine serum albumin is 1%, and the solution was further stirred for 30 min; after standing at 4° C. for 2 h, it was centrifuged at 1500 rpm for 15 min, the supernatant was removed and the pellet was discarded; the supernatant obtained was centrifuged at 12000 rpm for 30 min, and the supernatant was discarded, then 50 mL label-washing preservation solution was added and the resulting solution was centrifuged at 12000 rpm for 30 min again, the supernatant was discarded, and the precipitate obtained was resuspended with label-washing preservation solution to obtain the concentrated solution with the volume of 5.0 mL, which was stored at 4° C. for use, wherein the mass concentration of the nanogold-labeled anti-aflatoxin B₁ monoclonal antibody solution was 0.04 mg mL⁻¹;

Said 0.1 mol L⁻¹ potassium carbonate solution was obtained as follows: 13.8 g potassium carbonate was dissolved in purified water and diluted to 1000 mL of final volume, then filtered by 0.22 μm filter membrane; said 0.1 mg mL⁻¹ anti-aflatoxin B₁ monoclonal antibody solution was obtained as follows: 1 mg of commercially available anti-aflatoxin B₁ monoclonal antibody was diluted with water to 10 mL of final volume; said 10% bovine serum albumin aqueous solution was obtained as follows: 10 g bovine serum albumin was diluted with water to 100 mL, then filtered by 0.22 μm filter membrane; said label-washing preservation solution was obtained by mixing 2.0 g PEG-20000, 0.2 g sodium azide and 0.1235 g boric acid, to which water was added to 1000 mL, followed by filtration through 0.22 μm filter membrane.

(5) assembly of the multi-test-line digital immunochromatographic test strip for semi-quantitative detection of aflatoxin B₁

A water-absorbing pad, a detection pad, a gold-labeled pad and a sample pad were adhered sequentially on one surface of said paperboard from top to bottom, wherein each adjacent pads were overlapped and connected, and the length of each overlapped part was 2 mm, thus the multi-test-line digital immunochromatographic test strip for semi-quantitative detection of aflatoxin B₁ was prepared (see FIG. 2).

The method for use of the multi-test-line digital immunochromatographic test strip for semi-quantitative detection of aflatoxin B₁ is described as follows: the test samples of 1st and 2nd which had been grinded finely were weighed, to which methanol aqueous solution with the concentration of 60˜80% (v/v) was added, and the m/v ratio of the test sample and methanol solution was 2 g mL⁻¹. The solution was well-mixed and extracted with ultrasound at 60° C. water bath for 8 min, then left for 8 min. The supernatant (i.e. the extract) was diluted to 1:2.5 with water, resulting in that the final concentration of methanol in the dilution was 24%. 100 μL diluted sample solution used as the test solution was added dropwise to the sample pad of the multi-test-line digital immunochromatographic test strip for semi-quantitative detection of aflatoxin B₁. Said test strip was used as the test strip for detection. In the meantime, 100 μL of water was used as the negative control solution and added dropwise to the sample pad of another multi-test-line digital immunochromatographic test strip for semi-quantitative detection of aflatoxin B₁, which was used as the control test strip. The results are read out after 15 min.

Result evaluation: the test lines of the test strip for detection of the 1st test sample exhibited red lines, while the control line did not exhibit a red line, thus the result of this test strip for detection was considered as null, see FIG. 5-1: the test lines of the test strip for detection of the 1st test sample did not exhibit red lines, while the control line did not exhibit a red line either, thus the result of this test strip for detection was considered as null, see FIG. 5-2.

What is claimed is:

1. A test strip for digital detection of aflatoxin B₁ comprising:
a backing;
a sample pad,
a marker-labeled pad coated with a marker-labeled antibody against aflatoxin B₁;
a detection pad comprising a control line and a plurality of test lines, wherein the control line is coated with a secondary antibody against said marker-labeled antibody provided in said marker-labeled pad, and wherein said plurality of test lines are each coated with a different concentration of aflatoxin B₁-bovine serum albumin conjugate; and
a water-absorbing pad;
wherein all four pads are attached to one side of said backing with said water-absorbing pad on top, followed by said detection pad, said gold-labeled pad and said sample pad sequentially with said sample pad at the bottom, each pad in direct contact with its neighboring pad or pads.

2. The test strip of claim 1, wherein said backing comprises a paperboard:
said marker-labeled pad comprises a gold-labeled pad transversely coated with a nanogold-labeled anti-aflatoxin B₁ monoclonal antibody; and
said detection pad further comprises a nitrocellulose film on which said control line and said plurality of test lines are disposed in a direction perpendicular to a longitudinal axis of said test strip, and said plurality of test lines comprise a first test line (test line I), a second test line (test line II) and a third test line (test line III), test line I being the closest to said control line.

3. The test strip of claim 2, wherein the distances between the test line I, test line II and test line III on the detection pad and the upper border of the nitrocellulose film are 11˜17 mm, 13˜19 mm and 15˜21 mm, respectively, and the distances between each two adjacent test lines are at least 2 mm; and the distance between the control line and the test line I is 5˜11 mm.

4. The test strip of claim 2, wherein the coating amount of aflatoxin B₁-bovine serum albumin conjugate required per cm of the test line I, test line II and test line III on the detection pad are 120˜600 ng, 40˜200 ng, and 20˜100 ng, respectively.

5. The test strip of claim 2, wherein the particle diameter of the nanogold used in said gold-labeled pad is 15˜20 nm: and the amount of nanogold-labeled anti-aflatoxin B₁ monoclonal antibody required per cm of coating length on the gold-labeled pad is 60˜216 ng.

6. The test strip of claim 1, wherein said marker-labeled antibody against aflatoxin B₁ provided in said marker-labeled pad is derived from mouse and said secondary antibody is a rabbit anti-mouse polyclonal antibody.

7. The test strip of claim 6, wherein the coating amount of rabbit anti-mouse polyclonal antibody required per cm of the control line is 200˜500 ng.

8. The test strip of claim 1, wherein the water-absorbing pad has the length of 16˜18 mm and the width of 2˜4 mm; the detection pad has the length of 25˜30 mm and the width of 2˜4 mm; the marker-labeled pad has the length of 6˜9 mm and the width of 2˜4 mm; the sample pad has the length of 12˜18 mm and the width of 2˜4 mm, and overlapped parts of each adjacent pad have the length of 1˜3 mm.

9. A method for preparation of a test strip for aflatoxin B₁ comprises the following steps:
(1) preparation of a water-absorbing pad:
providing the water-absorbing pad by cutting a water-absorbing paper;
(2) preparation of a detection pad, comprising the following steps:
(a) coating a plurality of test lines:
preparing 0.1˜0.5 mg mL⁻¹ of coating solution A using aflatoxin B₁-bovine serum albumin conjugate; and coating a nitrocellulose film transversely with the coating solution A at about 11˜17 mm, 13˜19 mm and 15˜21 mm from the upper border of said film by spot-spraying, resulting in a test line I, test line II and test line III, the distances between each test line being at least 2 mm, wherein the coating amount of aflatoxin B₁-bovine serum albumin conjugate (AFB₁-BSA) required on per cm of the test line I, test line II and test line III is 120˜600 ng, 40˜200 ng, and 20˜100 ng, respectively, and
then drying said film for 8˜20 minutes at 37˜40° C.;
(b) coating a control line:
preparing 0.4˜0.6 mg mL⁻¹ of coating solution B using a rabbit anti-mouse polyclonal antibody; and coating the nitrocellulose film transversely with coating solution B 5˜11 mm from the test line I on the nitrocellulose film by spot-spraying to obtain the control line, wherein the coating amount of rabbit anti-mouse polyclonal antibody required on per cm of the control line is 200˜500 ng, and
then drying said film for 8˜20 minutes at 37˜40° C.;
(3) preparation of a sample pad:
soaking a fiberglass film is put into a blocking solution A, then taking out of the solution and drying for 10˜16 hours at 37˜40° C. to obtain the sample pad, and placing in a desiccator to store at room temperature;
(4) preparation of a gold-labeled pad:
spraying a nanogold-labeled anti-aflatoxin B₁ monoclonal antibody solution transversely onto the sample pad by spot-spraying, wherein the amount of nanogold-labeled anti-aflatoxin B₁ monoclonal antibody required per cm of spraying length is 60˜216 ng, then subjecting the coated pad to lyophilization under vacuum for 2˜6 hours, and placing in a desiccator to store at room temperature; and
(5) assembly of the test strip for semi-quantitative detection of aflatoxin B₁
attaching said water-absorbing pad, said detection pad, said gold-labeled pad and said sample pad sequentially on one side of a paperboard from top to bottom, such that each pad overlaps with adjacent pad or pads, the axial length of each overlapped part is 1˜3 mm.

10. The method of claim 9, wherein the coating solution A comprises 10˜50 mg of commercially available aflatoxin B₁-bovine serum albumin conjugate (AFB₁-BSA), 1˜2 g bovine serum albumin, 1˜2 g sucrose, 0.02˜0.05 g sodium azide, 0.8 g sodium chloride, 0.29 g disodium hydrogen phosphate dodecahydrate, 0.02 g potassium chloride, 0.02 g potassium dihydrogen phosphate, to which water is added to reach 100 mL of final volume; and
the coating solution B comprises 50 mg rabbit anti-mouse polyclonal antibody, 0.02˜0.05 g sodium azide, 0.8 g sodium chloride, 0.29 g disodium hydrogen phosphate dodecahydrate, 0.02 g potassium chloride, 0.02 g potassium dihydrogen phosphate, to which water is added to reach 100 mL of final volume.

11. The method of claim 9, wherein the blocking solution A comprises 1˜2 g bovine serum albumin, 0.1˜0.2 mL Triton X-100, 0.3 g polyvinylpyrrolidone, 2˜5 g sucrose, 0.02˜0.05 g sodium azide, 0.8 g sodium chloride, 0.29 g disodium hydrogen phosphate dodecahydrate, 0.02 g potassium chloride, 0.02 g potassium dihydrogen phosphate, to which water is added to reach 100 mL of final volume.

12. The method of claim 9, wherein the method for preparing said nanogold-labeled anti-aflatoxin B₁ monoclonal antibody solution comprises:
providing 50.0 mL of commercially available nanogold solution with the mass concentration of 0.01%, adjusting the pH of the solution to 5.5; adding slowly with stirring 2 mL of 0.1 mg mL⁻¹ anti-aflatoxin B₁ monoclonal antibody aqueous solution and stirring the solution for 30 min;
adding 10% (w/w) bovine serum albumin aqueous solution until the final concentration of bovine serum albumin is 1% (w/w), and further stirring the for 30 min;
after standing at 4° C. for 2 hours, centrifuging at 1500 rpm for 15 min, removing supernatant and discarding the pellet; and centrifuging the supernatant obtained at 12000 rpm for 30 min; after the supernatant is discarded, adding 50 mL label-washing preservation solution added and centrifuging the resulting solution at 12000 rpm for 30 min again; after the supernatant is discarded, resuspending the precipitate obtained with label-washing preservation solution to obtain the concentrated solution with the volume of 5 mL, which is stored at 4° C., wherein the mass concentration of the nanogold-labeled anti-aflatoxin B₁ monoclonal antibody solution is 0.04 mg mL⁻¹; and
preparing said label-washing preservation solution by mixing 2.0 g PEG-20000, 0.2 g sodium azide and 0.1235 g boric acid, to which water is added to 1000 mL, followed by filtration through 0.22 μm filter membrane.

13. Use of the test strip of claim 1 for semi-quantitative detection of aflatoxin B₁, wherein the test sample which has been grinded finely is weighed, to which methanol aqueous solution with the concentration of 60˜80% (v/v) is added, and the rrilv ratio of the test sample and methanol solution is 2 g mL⁻¹; the solution is well-mixed and extracted with sonication in 50˜60′ C. water-bath for 5˜10 min, then left for 5˜10 min, the supernatant (i.e. the extract) is diluted to 1:2.5 with water, resulting in that the final concentration of methanol in the dilution is 24˜32%; 100 μL diluted sample solution used as the test solution is added dropwise to the sample pad of the multi-test-line digital immunochromatographic test strip for semi-quantitative detection of aflatoxin B₁, and said test strip is used as the test strip for detection; in the meantime, 100 μL of water is used as the negative control solution and added dropwise to the sample pad of another multi-test-line digital immunochromatographic test strip for semi-quantitative detection of aflatoxin B₁, which is used as the control test strip; and the results are read out after 15 min; and
wherein result evaluation comprises: (1) positive: the control line of the test strip for detection of test sample shows a red line, and if the color of test line I among the three test lines is slightly lighter than that of the control test strip, and those of test line II and test line III are substantially the same as the control test strip, the content of aflatoxin B₁ in the sample is between 0.625 and 1.25 ng g⁻¹; if the test line I among the three test lines does not show red, and the colors of test line II and test line III are substantially the same as the control test strip, the content of aflatoxin B₁ in the sample is 1.25 ng g⁻¹; if the test line I among the three test lines does not show red, the color of test line II is lighter than that of the control test strip, and the color of test line III is substantially the same as the control test strip, the content of aflatoxin B₁ in the sample is between 1.25 and 2.5 ng g⁻¹; if the test line I and II of the three test lines do not show red, and the color of test line III is substantially the same as the control test strip, the content of aflatoxin B₁ in the sample is 2.5 ng g⁻¹; if the test line I and II of the three test lines do not show red, and the color of test line III is lighter than that of the control test strip, the content of aflatoxin B₁ in the sample is between 2.5 and 10 ng g⁻¹; if all the three test lines do not show red, the content of aflatoxin B₁ in the sample is not less than 10 ng g⁻¹; (2) negative: the control line of the test strip for detection of test sample shows a red line, and the colors of the three test lines are close to those of the control test strip, then it is a negative result, which indicates the content of aflatoxin B₁ in the sample is less than 0.625 ng g⁻¹; (3) null: no matter the test lines of the test strip for detection of test sample show red lines or not, the test strip is considered as null as long as the control line does not exhibit a red line.
